(19) United States
(12) Patent Application Publication
(10) Pub. No.: US 2007/0231344 A1
(43) Pub. Date: Oct. 4, 2007

(54) Conjugate vaccines for non-proteinaceous antigens

(75) Inventors: Elizabeth Leadbetter, Sharon, MA (US); Michael B. Brenner, Newton, MA (US); Gurdyal Singh Besra, Solihull (GB)

Correspondence Address: WOLF GREENFIELD & SACKS, P.C., 600 ATLANTIC AVENUE, BOSTON, MA, 02210-2206, US

(73) Assignees: The Brigham and Women's Hospital, Inc., Boston, MA (US); The University of Birmingham, Birmingham (GB)

(21) Appl. No.: 11/589,686

(22) Filed: Oct. 30, 2006

Related U.S. Application Data
(60) Provisional application No. 60/730,862, filed on Oct. 28, 2005.

(51) Int. Cl. A61K 39/385; A61K 39/09; A61K 39/095; A61K 39/112; A61K 39/145

(52) U.S. Cl. 424/193.1; 424/196.11; 424/197.11

(57) ABSTRACT

The present invention is directed to pharmaceutical compositions that can be used to immunize subjects using, for example, lipid, glycan, or nucleic acid antigens. These antigens are conjugated to a glycosphingolipid.

### RELATED APPLICATIONS

This application claims priority to U.S. Provisional Application Ser. No. 60/730862, entitled “CONJUGATE VACCINES FOR NON-PROTEINACEOUS ANTIGENS”, filed on Oct. 28, 2005, the entire contents of which are incorporated by reference herein.

### STATEMENT OF GOVERNMENT FUNDING

The United States Government has a paid-up license in this invention and the right in limited circumstances to require the patent owner to license others under reasonable terms as provided for by the terms of NIH Grant No. R37AI28973, awarded by the Department of Health and Human Services.

### FIELD OF THE INVENTION

The present invention is concerned with pharmaceutical compositions that can be used to induce an immune response in a subject. The compositions are characterized by the presence of a conjugate formed between an antigen, preferably a lipid or glycan, and a glycosphingolipid, preferably α-galactosylceramide. The compositions provide a strong and rapid response and may be used to either immunize subjects or to treat them for an ongoing infection or cancer.

### BACKGROUND OF THE INVENTION

Vaccination is believed by many to have contributed more to public health than any other medical procedure. It has been responsible for the elimination or near elimination of a number of devastating infectious diseases including smallpox, polio and diphtheria. Recently, attempts have been made to develop vaccines to treat other types of diseases, especially various types of cancer.

For the most part, vaccines have been based upon protein-derived antigens. However, work performed over the last decade has indicated that other types of antigens, particularly lipids, also play an important part in natural immunological defenses and could be exploited clinically (Dutronc et. al., Tissue Antigens 60:337-354 (2002)). From a therapeutic point of view, lipid antigens are particularly attractive because, unlike peptide antigens, they utilize a CD1 based pathway in which CD1 molecules are not polymorphic and thus are the same from person to person (Brigl and Brenner, Annu. Rev. Immunol. 22:817-90 (2004)). As a result, an immune treatment that is based upon lipids should work for essentially everyone.

Unfortunately, lipids tend to be only weakly immunogenic and methods for increasing their potency will need to be developed if lipid-based vaccines are to be successful. One traditional method for increasing the effectiveness of vaccines is to include an adjuvant. When lipid antigens are used, α-galactosylceramide (α-GalCer) has been reported to activate natural killer T cells in a manner that leads to an increased immunological response (U.S. Pat. No. 6,351,453; Burdin et al., J. Immunol. 161:3271-3281 (1998); Spada et al., J. Exp. Med. 188:1529-1534 (1998)). As a result, this compound and other glycosphingolipids have received a great deal of attention as potential agents for the prevention and treatment of cancer, autoimmune diseases and infectious diseases (Hokomori et al., J. Biochem. (Tokyo) 118:1091-1103 (1995); Hokomori, Glycoconj. J. 17:627-647 (200); Bektas et al., Glycoconj. J. 20:39-47 (2004); U.S. Pat. Nos. 6,635,622; 6,747,010; 6,555,372). Further improvements in this technology may lead to vaccines for diseases that have proven resistant to more traditional approaches.

### SUMMARY OF THE INVENTION

The present invention is based upon the concept that conjugates formed between certain glycosphingolipids (particularly α-GalCer) and antigens produce a strong and rapid immune response when administered to mice. This is reflected in the production of very high levels of antigen-specific IgM and IgG antibodies. The conjugated antigens produce a much stronger and more rapid effect than when antigen and α-GalCer are administered in an unconjugated form, i.e., the two components must be linked together. Immunization using the conjugates does not require the inclusion of adjuvants in vaccines and administration can be accomplished using a simple vehicle such as PBS.

In its first aspect, the invention is directed to a pharmaceutical composition, preferably a vaccine, comprising an antigen conjugated to a compound of Formula I:

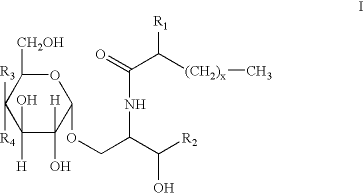

where: R₁ is H or OH;

R₂ is: ——CH₂(CH₂)_{Y}CH₃; —CH(OH)(CH₂)_{Y}CH₃; CH═CH(CH₂)_{Y}CH₃; or
——CH(OH)(CH₂)_{Y}CH(CH₃)CH₂CH₃, where Y=an integer from 5-17;

R₃ and R₄ are: H or OH such that when R₃ is H, R₄ is OH and when R₃ is OH, R₄ is H; and

X is an integer from 7-25.

Conjugation may occur at any carbon in the sugar ring of the structure of Formula I shown above and may involve either the direct binding of the antigen or attachment of antigen via a linker. This is illustrated by Formula II which in which antigen (Ag) is attached to the 2 carbon of the sugar (the preferred attachment site) by R₅.

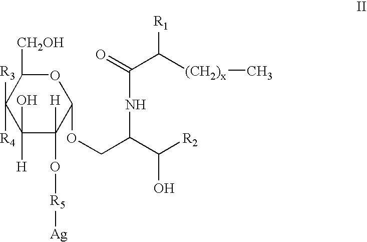

where R₁-R₄ are as defined above in connection with Formula I, Ag=antigen; and

R₅ is ——(CH₂)ₘ-(Z)ₚ(CH₂)_{q}——

and where: Z is O, S or N; m and q are independently an integer from 0-10; and p is an integer from 0-2.

It will be appreciated that the linkers described above as options for R₅ represent preferred embodiments and that other similar linkers should work equally well. Preferred options for R₅ occur when: a) m and p are 0, and q=2-10; and b) when m=1-3, Z=O, p=1, and q=1-7. To further illustrate conjugates, FIG. 1 shows a specific conjugate that was used in experiments. The antigen in this case, nitrophenyl, is shown within the box in the Figure.

The pharmaceutical compositions of the invention will include a pharmaceutically acceptable carrier such as PBS, saline or some other pharmaceutically acceptable vehicle.

The antigen may be a lipid, a glycan (i.e., mono or polysaccharide), a peptide or a nucleic acid antigen. In preferred embodiments, R₃ in Formula I is OH, R₂ is either ——CH(OH)(CH₂)_{Y}CH₃ or ——CH═CH(CH₂)_{Y}CH₃, with Y being 10-17. In other preferred embodiments, X=15-25 and a lipid antigen is conjugated to the sugar portion of the compound of Formula I, preferably at the 2 carbon. The most preferred compound of Formula I is α-GalCer.

Antigens for conjugation and use in vaccines include but are not limited to two general categories of molecules: bacterial polysaccharides and bacterial/viral glycolipids. Polysaccharides or oligosaccharides from Haemophilus influenza, Neisseria meningititidis, Salmonella typhi, Streptococcus pneumonia, Candida albicans, Cryptococcus neoformans, and Staphylococcus aureus, among others, have already been evaluated for their vaccine potential, and in many cases have been conjugated to protein carriers to improve their immunogenicity. (For an exhaustive review of these vaccines see An. Acad. Bras. Cienc. 77, no. 2, Rio de Janiero, June 2005.)

Alternatively, a compound of Formula I can be conjugated to bacterial or viral glycolipid components to create a vaccine specific for a glycolipid antigen. Recent papers have identified a number of serotype-specific cell surface glycolipids from Helicobacter pylori (Carbohydr. Res. 340(9):1605-1611 (2005)) and a secreted and membrane associated glycolipid antigen (GLXA) common to a number of Chlamydia strains (Curr. Microbiol. 46(3):217-23 (2003)) which are targeted by protective antibodies in murine models of disease. These are examples of a larger family of non-protein antigenic targets of bacteria and viruses which are appropriate candidates for conjugation and use in vaccines.

In addition to bacterial and viral antigens, conjugates may be made using antigens (including peptide, glycan or lipid antigens) derived from cancerous cells including those derived from the surface of cancerous cells. Unique glycolipids that have been shown to be possible tumor antigens, represent particularly good vaccine targets (see generally, Immunol. Cell Biol. 83(4):418-28 (2005)).

The invention also includes therapeutic packages containing any of the pharmaceutical compositions described above in a finished pharmaceutical container, preferably an injection ampoule, vial or syringe. When supplied in the form of an ampoule or vial, compositions may, optionally, be in a lyophilized or concentrated state. In these cases, it will be necessary to reconstitute or dilute preparations prior to administration to a subject and instructions for this should be included in the therapeutic package. The package may also, optionally, include the needed diluent or vehicle for reconstitution. In another embodiment, the therapeutic package may take the form of a prefilled disposable syringe containing enough pharmaceutical composition for administration to a single subject and, in all cases, the therapeutic packages should include instructions for the administration of the pharmaceutical composition to a subject for the purpose of inducing an immune response.

In one aspect, the invention provides a method for stimulating or inducing an immune response in a subject comprising administering to a subject in need thereof one or more of the pharmaceutical compositions described herein, in an amount effective to stimulate or induce an immune response.

In another aspect, the invention is directed to methods of inducing the production of antibodies against an antigen, particularly a lipid, glycan, peptide or nucleic acid antigen, by administering an effective amount of one or more of the pharmaceutical compositions described above. This method may be used prophylactically with an antigen derived from a source such as but not limited to a pathogenic virus, bacterium, fungus, mycobacterium or cancer cell for the purpose of immunizing the subject against future infection or disease. This method may also be ideal for a quarantine situation when an unusually rapid immune response is required. Preferred antigens will include those derived from the outer coat or membrane of a pathogenic agent or cell and those discussed above.

The conjugates may be combined with other buffers, salts or adjuvants as well as with other antigens which are either free or conjugated according to the invention. The conjugates may also be combined with the free form of antigens that are the same as those in the conjugate. The exact dose of antigen to be given to a subject and the schedule for administration, involving either single or multiple administrations, can be determined using methods that are standard in the art of immunology and medicine. The preferred route for administration in most cases will be by intramuscular, intravenous or subcutaneous injection but the invention is compatible with other routes of administration as well. In certain instances administration by inhalation, or by oral, or intranasal routes may be preferred.

In addition to the prophylactic administration of compositions as discussed above, conjugates may also be given as a treatment for subjects that have already been infected by a pathogenic agent or that have developed a disease. In these cases, administration should be initiated as soon as possible and continued until there is an improvement in one or more symptoms associated with the infection or disease. Instead of providing conjugates, it may be preferable in some instances to provide immune molecules, e.g., antibodies, to a subject from a different individual immunized with a conjugate of the invention. This “passive” transfer of immunity may be especially useful in subjects that have a compromised immune system, e.g., subjects taking immunosuppressive agents or HIV subjects.

Finally, it should be recognized that the compositions described above will be of value not only in the treatment of human subjects, but also in providing immunity to domestic animals or farm animals, and for the production of immune molecules such as antibodies from human and non-human (e.g., mice) subjects. Similarly, the compositions may be used in an experimental setting by researchers studying the immune system or attempting to develop a strategy for treating a particular disease or condition.

The invention provides, in yet another aspect, a method of synthesizing an antigen conjugate, comprising providing a compound of Formula III, 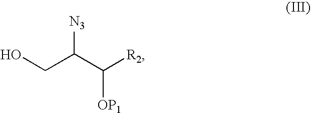
where R₂ is: ——CH₂(CH₂)_{Y}CH₃; —CH(OP₂)(CH₂)_{Y}CH₃; CH═CH(CH₂)_{Y}CH₃; or ——CH(OP₂)(CH₂)_{Y}CH(CH₃)CH₂CH₃, where Y=an integer from 5-17 and P¹ and P² can be the same or different and are hydrogen or a protecting group, or P¹ is joined with P² to form a cyclic protecting group; reacting the compound of Formula III with a monosaccharide optionally comprising protecting groups to form a covalent bond therebetween to form a compound of Formula IV, 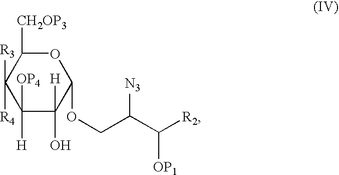
where R₃ and R₄ are H or OH, such that when R₃ is H, R₄ is OH and when R₃ is OH, R₄ is H, and P³ and P⁴ can be the same or different and are hydrogen or a protecting group; reacting the compound of Formula IV with an antigen to form covalent bond therebetween; and converting the azide group to an amide group and removing, if present, the protecting group(s) to form the antigen conjugate.

In one embodiment, the antigen conjugate is a compound of Formula I,

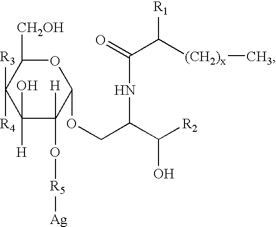

where: R₁ is H or OH;

R₂ is: ——CH₂(CH₂)_{Y}CH₃; —CH(OH)(CH₂)_{Y}CH₃; CH═CH(CH₂)_{Y}CH₃; or
——CH(OH)(CH₂)_{Y}CH(CH₃)CH₂CH₃, where Y=an integer from 5-17;

R₃ and R₄ are: H or OH such that when R₃ is H, R₄ is OH and when R₃ is OH, R₄ is H; and

X is an integer from 7-25.

The invention provides in another aspect, a method of synthesizing an antigen conjugate, comprising providing a compound of Formula (V), 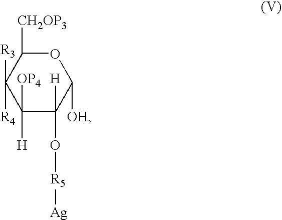
where Ag=antigen; R₃ and R₄ are H or OH, such that when R₃ is H, R₄ is OH and when R₃ is OH, R₄ is H; and R₅ is —(CH₂)ₘ-(Z)ₚ(CH₂)_{q}— where Z is O, S, or N; m and q can be the same or different and are integers from 0-10; p is an integer from 0-2; and P³ and P⁴ can be the same or different and are hydrogen or a protecting group; and reacting the compound of Formula V with an azide compound or an amide compound to form a covalent bond therebetween and removing, if present, the protecting group(s) to form the antigen conjugate.

In one embodiment, the azide compound is a compound of Formula (III),

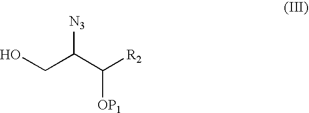

where R₂ is: —CH₂(CH₂)_{Y}CH₃, —CH(OP₂)(CH₂)_{Y}CH₃, CH═CH(CH₂)_{Y}CH₃, or —CH(OP₂)(CH₂)_{Y}CH(CH₃)CH₂CH₃, where Y=an integer from 5-17, and P¹ and P² can be the same or different and are hydrogen or a protecting group, or P¹ is joined with P² to form a cyclic protecting group.

In another embodiment, the amide compound is a compound of Formula (VI),

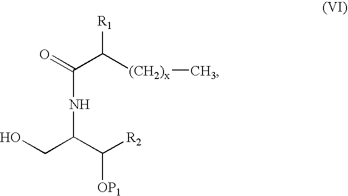

where R₁ is H or OH; R₂ is —CH₂(CH₂)_{Y}CH₃, —CH(OP₂)(CH₂)_{Y}CH₃, CH═CH(CH₂)_{Y}CH₃, or —CH(OP₂)(CH₂)_{Y}CH(CH₃)CH₂CH₃, where Y=an integer from 5-17, and P¹ and P² can be the same or different and are hydrogen or a protecting group, or P¹ is joined with P² to form a cyclic protecting group; and X is an integer from 7-25.

For the sake of brevity, these synthesis methods describe synthesis of conjugates in which the antigen is positioned at the 2 position of the sugar moiety. However, it is to be understood that the invention embraces synthesis methods in which the antigen is present at other positions (e.g., other positions in the sugar moiety) and the modifications required for such syntheses will be understood by one of ordinary skill in the art.

These and other aspects of the invention will be described in greater detail below. Throughout this disclosure, all technical and scientific terms have the same meaning as commonly understood by one of ordinary skill in the art to which this invention pertains unless defined otherwise.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 shows the conjugate NP-α-GalCer. The NP (nitrophenyl) group is shown in the box and is attached to the 2 carbon of the galactose ring.

FIG. 2 shows an illustrative embodiment for the synthesis of antigen conjugates.

FIG. 3 shows an illustrative embodiment for the synthesis of antigen conjugates.

### DEFINITIONS

The following definitions are provided for the purpose of comprehension of the present invention but are not meant to be limiting.

Adjuvant: The term “adjuvant” as used herein refers to non-specific stimulators of the immune response. Unconjugated α-GalCer has been used in the past to promote an immune response when lipid antigens are used. Examples of other adjuvants that have been employed, primarily with proteinaceous antigens, include complete and incomplete Freund's adjuvant, aluminum hydroxide, modified muramyldipeptide mineral gels such as aluminum hydroxide, surface active substances such as lysolecithin, pluronic polyols, polyanions, peptides, immunostimulatory nucleic acids (e.g., CpG nucleic acids), oil emulsions, keyhole limpet hemocyanins, dinitrophenol, saponins, BCG (bacille Calmette-Guerin) and Corynebacterium parvum. If desired, any of these adjuvants may be included in the vaccine compositions disclosed herein in an attempt to further boost an immune response. However, an advantage of the disclosed conjugates is that a strong immune response is generated using the conjugate alone, i.e., in the absence of added adjuvant. Thus, the addition of adjuvant to compositions is optional.

Patient or Subject: The present compositions and methods can be used in humans, domestic animals (cats or dogs), zoo animals and farm animals (cattle, sheep, horses, pigs, etc.), but are not so limited. It will be understood that the terms “patient” and “subject” include all of these species unless context indicates otherwise.

Antibody: Antibody embraces monoclonal antibodies, polyclonal antibodies, and antibodies from different species. It also embraces single chain antibodies as well as fully formed four chain antibodies. Antibody fragments mean a fragment, preferably an antigen-binding fragment, of an antibody including but not limited to Fv, Fab, F(ab)₂ fragments. The antibody may be a chimeric antibody such as a humanized antibody.

Active immunization: The term “active immunization” refers to the induction of an immune response in an individual elicited by the administration of an immunogen, vaccine, antigen or, in the context of the invention, a conjugate of a compound of Formula I and an antigen.

Antigen: As used herein, the term “antigen” refers to a molecule capable of inducing a humoral immune response and/or a cellular immune response leading to the activation of B- and/or T-lymphocytes upon administration to a subject. This may however require that, as discussed herein, the antigen be conjugated to a compound of Formula I.

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Bound or Conjugated: As used herein, the term “bound” or “conjugated” refers to binding or attachment that may be covalent, e.g., by chemical coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like.

Conjugate: As used herein, the noun “conjugate” refers to a compound of Formula I joined by one of the carbons in its sugar ring (e.g., the carbon in the 2 position) to an antigen (e.g., a glycan, lipid or nucleic acid). Conjugates may be chemically synthesized with the antigen as part of the structure.

Effective Amount or Therapeutically Effective Amount: As used herein, these terms refer to an amount of a composition sufficient to achieve a desired biological effect. For example, depending on the context, an effective amount may be an amount sufficient to induce an immune response or to alleviate one or more disease symptoms.

Isolated: As used herein, the term “isolated” refers to a molecule that has been removed from its native environment. For example, an antibody or antigen present in a living animal is not “isolated,” but the same antibody or antigen separated from the materials of its natural state, e.g., purified by some biochemical procedure, is “isolated.”

Immune response: As used herein, the term “immune response” refers to a humoral immune response and/or cellular immune response leading to the activation or proliferation of B- and/or T-lymphocytes and/or antigen presenting cells. “Immunogenic” refers to an agent used to stimulate the immune system of a living organism. One or more functions of the immune system are increased and may be directed towards the immunogenic agent.

Immunization: As used herein, the terms “immunize” or “immunization” or related terms refer to conferring the ability to mount a substantial immune response (comprising antibodies and/or cellular immunity) against a target antigen. These terms do not require that complete immunity be created, but rather that a measurable immune response be produced when the target antigen is administered.

Passive immunization: As used herein, the term “passive immunization” refers to conferral of immunity by the administration, by any route, of exogenously produced immune molecules (e.g., antibodies) or cells (e.g., T cells) into a subject. Passive immunization differs from “active” immunization, where immunity is obtained by introduction of an immunogen, vaccine, antigen or conjugate into an individual to elicit an immune response.

Purified: When the term “purified” is used in reference to a molecule, it means that the concentration of the molecule has been increased relative to molecules associated with it in its natural environment, or the environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be at least 20%, 40%, 60%, 80%, 90%, 95% or 99% pure when considered relative to its contaminants.

Vaccine: As used herein, the term “vaccine” refers to a formulation which contains an antigen and which is in a form that is capable of being administered to a subject animal. The vaccine may contain the antigen in a conjugated form, as described herein. Typically, the vaccine will include a pharmaceutically acceptable carrier, i.e., a composition in which antigens or conjugates are dissolved, mixed, suspended or emulsified and which can be safely administered to a subject. Upon introduction into a host, the vaccine will provoke an immune response including, but not limited to, the production of antibodies and/or cytokines and/or the activation of cytotoxic T cells, antigen presenting cells, helper T cells, dendritic cells and/or other cellular responses. Optionally, a vaccine of the present invention may include an adjuvant.

### DETAILED DESCRIPTION OF THE INVENTION

The conjugates described herein are effective in inducing a rapid and strong immune response. Thus, they are useful in compositions for the immunization of humans and animals either therapeutically (after symptoms become apparent) or prophylactically against diseases, particularly cancer or infections. Antibodies produced by immunization with the conjugates are also useful for therapeutic and prophylactic purposes.

In one aspect, the invention provides a method for stimulating or inducing an immune response in a subject comprising administering to a subject in need thereof one or more of the pharmaceutical compositions described herein, in an amount effective to stimulate or induce an immune response. The immune response can be a humoral immune response or a cell-mediated immune response. It may be local or systemic, usually depending upon the route of administration. Thus, the immune response may be measured by the quality or quantity of antibody production, the production and/or synthesis of cytokines such as IFN-gamma as well as others, the activation of cell types including but not limited to B cells, T cells, NK cells as well as others, and the like. The immune response may be beneficial to a subject even if alone it is not curative of the condition from which the subject suffers. For example, it may help to reduce some symptoms or to provide relief in the short term.

It is to be understood that the conjugates and compositions of the invention have uses in addition to therapeutic treatment of human subjects. For example, they can be used to make antibodies in normal humans, or in experimental animals such as mice and/or rabbits.

Synthesis of Conjugates

It will be understood that the term “conjugates” as used herein refers to compounds in which antigen is joined to a compound of Formula I regardless of how the linking is accomplished. The most preferred method is to use standard chemical methods to synthesize a conjugate with the antigen as part of the structure in a selected and specific position (preferably at the 2 carbon) on the sugar.

The present invention provides methods for the synthesis of antigen conjugates. In some cases, the method comprises attachment (e.g., via glycosylation) of an azide compound to a species comprising a monosaccharide moiety covalently bonded to at least one lipid and an azide group. The species may then be reacted with an antigen precursor such that a covalent bond is formed therebetween. The covalent bond may be formed between the antigen and the species, for example, via the monosaccharide portion of the species. For example, the antigen may form a bond to a six-membered monosaccharide via the 2′-, 4′-, or 6′-position of the ring, but its placement is not so limited. Conversion of the azide compound to an amide compound may then produce the antigen conjugate.

In some cases, the method may comprise attaching a compound comprising a monosaccharide moiety covalently bonded to an antigen to an amide compound to form the antigen conjugate. In other cases, the compound may be attached to an azide compound, wherein subsequent conversion of the azide moiety to an amide moiety may then produce the antigen conjugate.

Protecting groups may optionally be utilized in the synthesis of antigen conjugates. As used herein, a “protecting group” refers to temporary substituents which protect a potentially reactive functional group from undesired chemical transformations. Examples of such protecting groups include, but are not limited to, esters of carboxylic acids, esters of alcohols (e.g., acetyl groups, and the like), silyl ethers of alcohols including t-butyldimethyl silyl (TBDMS) groups, trimethylsilyl (TMS) groups, and the like, and acetals and ketals of aldehydes and ketones, respectively. In some cases, the protecting group is a cyclic protecting group. Other examples of protecting groups, as well as methods for protection and deprotection, are described in, Greene, T. W.; Wuts, P. G. M. Protective Groups in Organic Synthesis, 2^{nd} ed.; Wiley: New York. 1991.

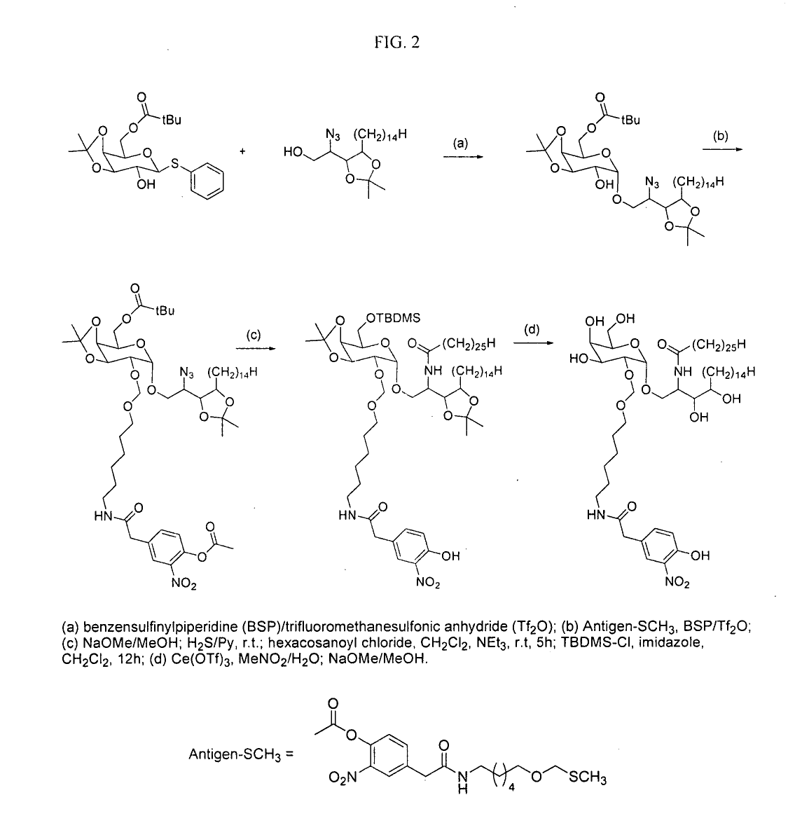

For example, the method may comprise providing a compound of Formula III, 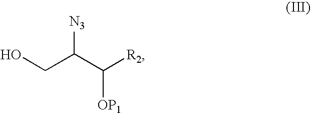
where R₂ is: —CH₂(CH₂)_{Y}CH₃; —CH(OP₂)(CH₂)_{Y}CH₃; —CH═CH(CH₂)_{Y}CH₃; or —CH(OP₂)(CH₂)_{Y}CH(CH₃)CH₂CH₃, where Y=an integer from 5-17 and P¹ and P² can be the same or different and are hydrogen or a protecting group, or P¹ is joined with P² to form a cyclic protecting group. The compound may then be reacted with a monosaccharide optionally comprising protecting groups to form a covalent bond therebetween to form a compound of Formula IV, 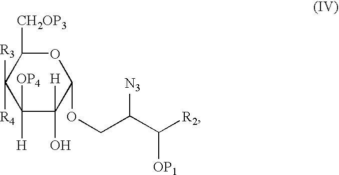
where R₃ and R₄ are H or OH, such that when R₃ is H, R₄ is OH and when R₃ is OH. R₄ is H, and P³ and P⁴ can be the same or different and are hydrogen or a protecting group. The compound of Formula IV may then be reacted with an antigen to form covalent bond therebetween, and conversion of the azide group to an amide group and removal of any protecting group(s), if present, may produce the antigen conjugate. FIG. 2 shows an illustrative embodiment for the synthesis of antigen conjugates.

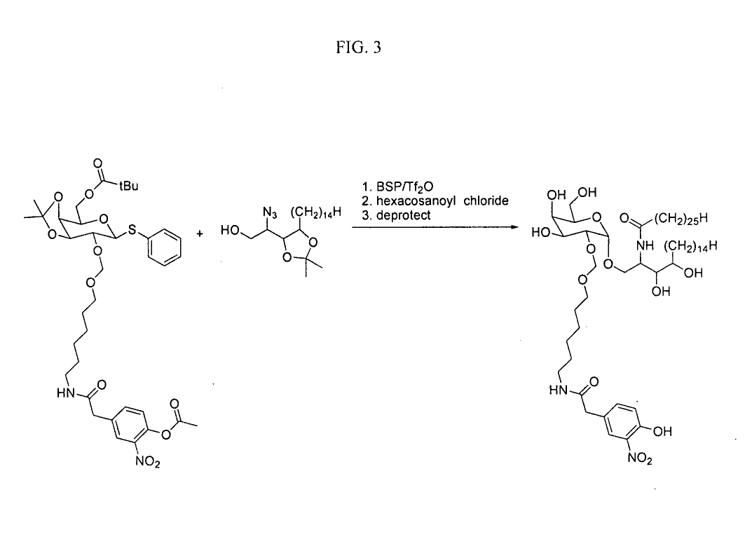

In another embodiment, the method may comprise providing a compound of Formula (V), 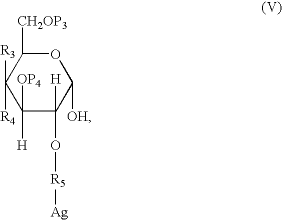
where Ag=antigen; R₃ and R₄ are H or OH, such that when R₃ is H, R₄ is OH and when R₃ is OH, R₄ is H; and R₅ is —(CH₂)ₘ-(Z)ₚ(CH₂)_{q}— where Z is O, S, or N; m and q can be the same or different and are integers from 0-10; p is an integer from 0-2; and P³ and P⁴ can be the same or different and are hydrogen or a protecting group. The compound of Formula V may then be reacted, i.e., via glycosylation, with an azide compound or an amide compound to form a covalent bond therebetween. Removal of the protecting group(s), if present, may then form the antigen conjugate. The azide group may be a compound of Formula III, as described herein. The amide compound may be a compound of Formula (VI), 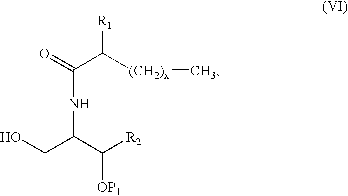
where R₁ is H or OH; R₂ is —CH₂(CH₂)_{Y}CH₃, —CH(OP₂)(CH₂)_{Y}CH₃, CH═CH(CH₂)_{Y}CH₃, or —CH(OP₂)(CH₂)_{Y}CH(CH₃)CH₂CH₃, where Y=an integer from 5-17, and P¹ and P² can be the same or different and are hydrogen or a protecting group, or P¹ is joined with P² to form a cyclic protecting group; and X is an integer from 7-25. FIG. 3 shows an illustrative embodiment for the synthesis of antigen conjugates.

An alternative procedure is to separately synthesize the compound of Formula I and the antigen and then join them together. For example, the compound of Formula I may be synthesized with one carbon position on the sugar (the one where antigen is to attach) occupied by a reactive group and all other possible antigen attachment sites occupied by blocking groups. The antigen may then be synthesized with a complementary reactive group to facilitate attachment. After the antigen is joined, the blocking groups on the compound may be removed using standard methodology. Finally, it is possible to synthesize the compound of Formula I and then attach an antigen that has been obtained from a natural source rather than by synthesis. In all cases, antigen may be either joined directly to the sugar or joined via a linker (e.g., an aliphatic chain of 2-10 carbons, and other linkers as described herein).

The compounds of Formula I, including the most preferred compound, α-GalCer, may be synthesized using processes known in the art (see U.S. Pat. Nos. 5,767,092, 5,780,441, 5,849,716, 5,936,076, 6,417,167, 6,531,453, 6,555,372, 6,635,622, and 6,747,010, the teachings of which are all hereby incorporated by reference). The invention contemplates the conjugation of antigens to any of the immunostimulatory sphingoglycolipid compounds including other α-GalCer derivatives described in the afore-mentioned references.

The conjugates of the invention may be those in which a compound of Formula I is joined to a glycan, lipid or nucleic acid antigen. Peptide antigens may also be employed if desired and used either alone or in combination with another antigen. Many methods for conjugating peptides have been described in the art and, in some instances may be adapted to the conjugation of other antigens as well (see e.g., Sambrook et al., eds., Molecular Cloning A Laboratory Manual, 2nd edition, Cold Spring Harbor Laboratory Press, Cold Spring Harbor, N.Y. (1989); Ausubel et al., eds., Current Protocols in Molecular Biology, John H. Wiley & Sons, Inc. (1997); Celis, ed., Cell Biology, Academic Press, 2nd edition (1998); Harlow et al., Antibodies. A Laboratory Manual, Cold Spring Harbor Laboratory, Cold Spring Harbor, N.Y. (1988), all of which are incorporated herein by reference in their entireties.)

Methods of attaching peptide antigens may involve the use of heterobifunctional cross-linkers. These include the cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available, for example from the Pierce Chemical Company (Rockford, Ill., USA), and having one functional group reactive towards amino groups and one functional group reactive towards SH residues. The above mentioned cross-linkers all lead to formation of a thioether linkage. Another class of cross-linkers that might be used in the practice of the invention is characterized by the introduction of a disulfide linkage between the linked components. Cross-linkers belonging to this class include for example SPDP and Sulfo-LC-SPDP (Pierce).

Peptide antigens may also be coupled by cross-linking using carbodiimide compounds. These include the carbodiimide EDC (1-(3-dimethylaminopropyl)-3-ethylcarbodiimide hydrochloride), which can optionally also be used with N-hydroxy-succinimide NHS in the reaction. Additional cross-linking methods and cross-linkers, suitable for attaching antigens as well as guidance on performing the coupling reactions and on the use of chemical cross-linkers can be found in Hermanson, Bioconjugate Techniques, Academic Press Inc., San Diego, Calif., USA.

Other methods of forming conjugates may involve biotinylating one component, i.e., either the antigen or the compound of Formula I, and linking the other component to avidin or streptavidin. Many variations of this procedure may be used as will be readily apparent to those of skill in the art. For example, both components may be biotinylated and then coupled with a streptavidin or avidin linker.

Agents of Formula I (and alternatives thereto) can be conjugated antigens using any mechanism known in the art. There are many standard techniques known to those of ordinary skill in the art. For instance, functional groups which are reactive with various labels include, but are not limited to, (functional group: reactive group of light emissive compound) activated ester:amines or anilines: acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones: imido ester:amines or anilines; isocyanate:amines or anilines; and isothiocyanate:amines or anilines.

Examples of crosslinkers useful for nucleic acids include furocoumarins (e.g., haloalkyl furocoumarin and haloalkyl coumarin), benzodipyrones (e.g., haloalkyl benzodipyrone), bis-azides (e.g., bis-azido ethidium bromide and azido nucleoside triphosphate), and psoralens (e.g., 8-methoxypsoralen, 5-methoxypsoralen, 4,5′,8-trimethylpsoralen, 4′-hydroxymethyl -4,5′,8-trimethylpsoralen and 4′-aminomethyl-4,5′,8-trimethylpsoralen). Other crosslinkers are provided in U.S. Pat. No.4,599,303, issued on Jul. 8, 1996. Covalent conjugation of end-labeled nucleic acids to peptides can be accomplished by SPDP-SATA crosslinking.

Linkers can be any of a variety of molecules, preferably nonactive, such as nucleotides or multiple nucleotides, straight or even branched saturated or unsaturated carbon chains of C₁-C₃₀, phospholipids, amino acids, and in particular glycine, and the like, whether naturally occurring or synthetic. Additional linkers include alkyl and alkenyl carbonates, carbamates, and carbamides. These are all related and may add polar functionality to the linkers such as the C₁-C₃₀ previously mentioned. As used herein, the terms linker and spacer are used interchangeably. The length of the spacer can vary depending upon the application and the nature of the components being conjugated.

Linkers or spacers may be homo-bifunctional or hetero-bifunctional cross-linkers, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl)suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl [4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide.2 HCl, 4-(4-N-maleimidophenyl)-butyric acid hydrazide.2 HCl, and 3-[2-pyridyldithio]propionyl hydrazide. The cross-linkers are bis-[β-4-azidosalicylamido)ethyl]disulfide and glutaraldehyde.

Other cross linkers include psoralen, furocoumarins, benzodipyrones and bis-azides, as described herein.

Amine or thiol groups may be added at any nucleotide of a nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Preparation of Lipid or Glycan Antigens

Methods for selecting and preparing antigens for the compositions and methods of treatment described herein are known to those skilled in the art. Representative examples of such antigens include but are not limited to antigens derived from the coat or outer membrane of a pathogenic bacterium, mycobacterium or virus; a lipid, glycan or peptide derived from a cell, e.g. a cancer cell; or a synthetic molecule. In cases where a specific antigenic determinant is known this may be used in coupling reactions and, for the purposes of the present invention will be considered as the “antigen” in these cases.

Antigens include, but are not limited to, proteins, polypeptides, peptides, polysaccharides such as glycans, polysaccharide conjugates, peptide and non-peptide mimics of polysaccharides and other molecules, small molecules, lipids, glycolipids, and carbohydrates. Methods for administering an antigen to a subject are well known in the art. In general, an antigen is administered directly to the subject by any means, such as, e.g., parenteral administration such as intravenous, intramuscular, or subcutaneous administration, transdermal, mucosal, intranasal, or intratracheal administration or oral administration. The antigen can be administered systemically or locally.

Vaccines and Their Use

The invention provides compositions which may be used for stimulating immune responses in human and other subjects, which may be beneficial for but is not limited to preventing and/or treating diseases. As used herein, to treat a subject means to provide some therapeutic or prophylactic benefit to the subject. This may occur by reducing partially or completely symptoms associated with a particular condition. Treating a subject is not however limited to curing the subject of the particular condition.

In one embodiment, the compositions stimulate an immune response leading to the production of immune molecules, including antibodies, that bind to antigens. The invention comprises vaccines sufficient to reduce the number, severity and/or duration of symptoms. The vaccines may also contain antigens in free form and such antigens may be the same as or different from those in the conjugate.

In addition to the conjugates, a vaccine may include salts, buffers, adjuvants and other substances, or excipients which may be desirable for improving its efficacy. Examples of suitable vaccine components as well as a general guidance with regard to methods for preparing effective compositions may be found in standard texts such as Remington's Pharmaceutical Sciences (Osol, A, ed., Mack Publishing Co., (1990)). In all cases, conjugates should be present in an “effective amount” (i.e., an amount that produces the desired physiological effect) and other components of the vaccine should be physiologically acceptable.

The antigenic preparations can be administered by either single or multiple dosages of an effective amount of conjugate. Effective amounts of the compositions of the invention can be determined for different antigens using methods that are standard in the art of immunology. Typically, it is expected that an effective dose based upon the weight of the antigen will vary from 0.01 to 5,000 μg/ml per dose, more typically from 0.1 to 500 μg/ml per dose, and most typically from 10 to 300 μg/ml per dose. Rough guidance may be obtained in particular cases by reference to existing vaccines. For example, capsular polysaccharide conjugate vaccines for Streptococcus pneumonia and Haemophilus influenzae type b (Hib) are in widespread use. The recommended doses for the two S. pnuemo vaccines (PneumoVax and Prevnar) are listed in the Complete Drug Reference physician's manual as 0.5 ml of Pneumovax (contains 25 ug each of 23 different polysaccharides) to be given subcutaneously or intramuscularly or 0.5 ml of Prevnar (contains only 7 polysaccharides) to be given intramuscularly. Each vaccine is administered 4 times over prescribed intervals. Other vaccinations that may be modified using the compositions and methods of the invention include Polio, DTaP, MMR, Varicella, DPT, tetanus-diphtheria (i.e., Td), and Hepatitis B vaccine.

Vaccines may be administered to subjects by any route known in the art, including parenteral routes (e.g., injection), inhalation, topical or by oral administration. The preferred method is by intramuscular, intravenous, or subcutaneous injection. Suitable carriers that may be used in preparations for injection include sterile aqueous (e.g., physiological saline) or non-aqueous solutions and suspensions such as propylene glycol, polyethylene glycol, vegetable oils such as olive oil, and injectable organic esters such as ethyl oleate. Treatment and dosing strategies may be developed using guidance provided by standard reference works (see e.g., N. Engl. J. Med. 345(16):l 177-83 (2001) for treatment of children; and Arch. Intern. Med. 154(22):2545-57 (1994) for adult treatments; see, Arch. Intern. Med. 28; 154(4):373-7 (1994) for a review of clinical trials covering a fifteen year span of vaccine use).

Because the conjugates described herein act rapidly to induce an immune response, vaccines may be administered to a subject to treat an infection or disease after symptoms have appeared. In these cases, it will be advantageous to initiate treatment as soon after the onset of symptoms as possible and, depending on circumstances, to combine vaccine administration with other treatments, e.g., the administration of an antibiotic. Examples of antibiotics are provided below. The immune molecules produced by immunization with vaccines, e.g., antibodies, may also be useful when transferred from an immunized individual to another individual not immunized, thus “passively” transferring immunity. This procedure may be employed in infected subjects in cases where a subject is immuno-compromised, or in cases where, despite the rapid effect of the conjugates described herein, there is insufficient time for a subject to mount their own immune response against a pathogen.

Antigens and Sources

The antigens may be derived from bacteria, mycobacteria, viruses, fungi and parasites. It is to be understood that antigens derived from a particular microorganism can be used to prevent and/or treat an infection of that microorganism. Below is provided a list of microorganisms from which antigens may be derived.

Bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacilliis moniliformis, Treponema pallidium, Treponema pertenue, Leptospira, Rickettsia, and Actinomyces israelli.

Viruses include, but are not limited to, interoviruses (including, but not limited to, viruses that the family picornaviridae, such as polio virus, coxsackie virus, echo virus), rotaviruses, adenovirus, hepatitus. Specific examples of viruses that have been found in humans include but are not limited to Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Viruses that infect both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-I, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1. Sendai virus, Hemadsorption virus, Parainifluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include but are not limited to the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A,B,C,D,E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans (an organism which is normally part of the respiratory tract flora), cryptococcus neoformans (sometimes in normal flora of respiratory tract), and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Fungi include but are not limited to microsporum or traicophyton species, i.e., microsporum canis, microsporum gypsum, tricofitin rubrum, Cryptococcus neoformans, Histoplasma capsulatum, Coccidioides immitis, Blaslomyces dermatitidis, Chlamydia trachomatis, and Candida albicans.

Parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative, and is not intended to be limiting.

The antigen may be derived from microorganisms that can be used as biowarfare agents. Examples of microorganisms are listed below.

CDC Category A agents include Bacillus anthracis (otherwise known as anthrax), Clostridium botulinum and its toxin (causative agent for botulism), Yersinia pestis (causative agent for the plague), variola major (causative agent for small pox), Francisella tularensis (causative agent for tularemia), and viral hemorrhagic fever causing agents such as filoviruses Ebola and Marburg and arenaviruses such as Lassa, Machupo and Junin.

CDC Category B agents include Brucellosis (Brucella species), epsilon toxin of Clostridium perfringens, food safety threats such as Salmonella species, E. coli and Shigella, Glanders (Burkholderia mallei), Melioidosis (Burkholderia pseudomallei), Psittacosis (Chlamydia psittaci), Q fever (Coxiella burnetii), ricin toxin (from Ricinus communis—castor beans), Staphylococcal enterotoxin B, Typhus fever (Rickettsia prowazekii), viral encephalitis (alphaviruses, e.g., Venezuelan equine encephalitis, eastern equine encephalitis, western equine encephalitis), and water safety threats such as e.g., Vibrio cholerae, Cryptosporidium parvum.

CDC Category C agents include emerging infectious diseases such as Nipah virus and hantavirus.

Other pathogens that can be detected using the methods of the invention include Gonorrhea, H. pylori, Staphylococcus spp., Streptococcus spp. such as Streptococcus pneumoniae, Syphilis; viruses such as SARS virus, Hepatitis virus, Herpes virus, HIV virus, West Nile virus, Influenza virus, poliovirus, rhinovirus; parasites such as Giardia, and Plasmodium malariae (malaria); and mycobacteria such as M. tuberculosis.

Antigens useful in the invention may include toxins or other molecules produced from microorganisms. Examples of such molecules are provided below.

Examples of toxins include abrin, ricin and strychnine. Further examples of toxins include toxins produced by Corynebacterium diphtheriae (diphtheria), Bordetella pertussis (whooping cough), Vibrio cholerae (cholera), Bacillus anthracis (anthrax), Clostridium botulinum (botulism), Clostridium tetani (tetanus), and enterohemorrhagic Escherichia coli (bloody diarrhea and hemolytic uremic syndrome), Staphylococcus aureus alpha toxin, Shiga toxin (ST), cytotoxic necrotizing factor type 1 (CNF1), E. coli heat-stable toxin (ST), botulinum, tetanus neurotoxins, S. aureus toxic shock syndrome toxin (TSST), Aeromonas hydrophila aerolysin, Clostridium perfringens perfringolysin O, E. coli hemolysin, Listeria monocytogenes listeriolysin O, Streptococcus pneumoniae pneumolysin, Streptococcus pyogenes streptolysine O, Pseudomonas aeruginosa exotoxin A, E. coli DNF, E. coli LT, E. coli CLDT, E. coli EAST, Bacillus anthracis edema factor, Bordetella pertussis dermonecrotic toxin, Clostridium botulinum C2 toxin, C. botulinum C3 toxin, Clostridium difficile toxin A, and C. difficile toxin B.

Further examples of bacteria that can be used as biohazards include but are not limited to Streptococcus spp., Staphylococcus spp., Pseudomonas spp., Clostridium difficile, Legionella spp., Pneumococcus spp., Haemophilus spp. (e.g., Haemophilus influenzae), Klebsiella spp., Enterobacter spp., Citrobacter spp., Neisseria spp. (e.g., N. meningitidis, N. gonorrhoeae), Shigella spp., Salmonella spp., Listeria spp. (e.g., L. monocytogenes), Pasteurella spp. (e.g., Pasteurella multocida), Streptobacillus spp., Spirillum spp., Treponema spp. (e.g., Treponema pallidum), Actinomyces spp. (e.g., Actinomyces israelli), Borrelia spp., Corynebacterium spp., Nocardia spp., Gardnerella spp. (e.g., Gardnerella vaginalis), Campylobacter spp., Spirochaeta spp., Proteus spp., Bacteriodes spp., H. pylori, and anthrax.

Further examples of viruses that can be used as biohazards include but are not limited to HIV, Herpes simplex virus 1 and 2 (including encephalitis, neonatal and genital forms), human papilloma virus, cytomegalovirus, Epstein Barr virus, Hepatitis virus A, B and C, rotavirus, adenovirus, influenza A virus, respiratory syncytial virus, varicella-zoster virus, small pox, monkey pox and SARS virus.

Further examples of fungi that can be used as biohazards include but are not limited to candidiasis, ringworm, histoplasmosis, blastomycosis, paracoccidioidomycosis, crytococcosis, aspergillosis, chromomycosis, mycetoma, pseudallescheriasis, and tinea versicolor.

Further examples of parasites that can be used as biohazards include but are not limited to protozoa and nematodes such as amebiasis, Trypanosoma cruzi, Fascioliasis (e.g., Facioloa hepatica), Leishmaniasis, Plasmodium (e.g., P. falciparum, P. knowlesi, P. malariae,)Onchocerciasis, Paragonimiasis, Trypanosoma brucei, Pneumocystis (e.g., Pneumocystis carinii), Trichomonas vaginalis, Taenia, Hymenolepsis (e.g., Hymenolepsis nana), Echinococcus, Schistosomiasis (e.g., Schistosoma mansoni), neurocysticercosis, Necator americanus, and Trichuris trichuria.

Further examples of pathogens that can be used as biohazards include but are not limited to Chlamydia, M. tuberculosis, and M. leprosy, and Rickettsiae.

An antigen that can be used in subjects having or at risk of developing cancer includes but is not limited to a cancer antigen. Cancer antigens include but are not limited to HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27. HMFG-2. SM-3, B72.3. PR5C5, PR4D2, and the like. Other cancer antigens are described in U.S. Pat. No. 5,776,427.

Cancer antigens can be classified in a variety of ways. Cancer antigens include antigens encoded by genes that have undergone chromosomal alteration. Many of these antigens are found in lymphoma and leukemia. Even within this classification, antigens can be characterized as those that involve activation of quiescent genes. These include BCL-1 and IgH (Mantel cell lymphoma), BCL-2 and IgH (Follicular lymphoma), BCL-6 (Diffuse large B-cell lymphoma), TAL-1 and TCR□ or SIL (T-cell acute lymphoblastic leukemia), c-MYC and IgH or IgL (Burkitt lymphoma), MUN/IRF4 and IgH (Myeloma), PAX-5 (BSAP) (Immunocytoma).

Other cancer antigens that involve chromosomal alteration and thereby create a novel fusion gene and/or protein include RAR□, PML, PLZF, NPM or NuMA (Acute promyelocytic leukemia), BCR and ABL (Chronic myeloid/acute lymphoblastic leukemia), MLL (HRX) (Acute leukemia), E2A and PBX or HLF (B-cell acute lymphoblastic leukemia), NPM, ALK (Anaplastic large cell leukemia), and NPM, MLF-1 (Myelodysplastic syndrome/acute myeloid leukemia).

Other cancer antigens are specific to a tissue or cell lineage. These include cell surface proteins such as CD20, CD22 (Non-Hodgkin's lymphoma, B-cell lymphoma, Chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (Epithelial and lymphoid malignancies), Human leukocyte antigen (HLA)-DR, HLA-DP, and HLA-DQ (Lymphoid malignancies), RCAS1 (Gynecological carcinomas, bilary adenocarcinomas and ductal adenocarcinomas of the pancreas), and Prostate specific membrane antigen (Prostate cancer).

Tissue- or lineage-specific cancer antigens also include epidermal growth factor receptors (high expression) such as EGFR (HER1 or erbB1) and EGFRvIII (Brain, lung, breast, prostate and stomach cancer), erbB2 (HER2 or HER2/neu) (Breast cancer and gastric cancer), erbB3 (HER3) (Adenocarcinoma), and erbB4 (HER4) (Breast cancer).

Tissue- or lineage-specific cancer antigens also include cell-associated proteins such as Tyrosinase, Melan-A/MART-1, tyrosinase related protein (TRP)-1/gp75 (Malignant melanoma), Polymorphic epithelial mucin (PEM) (Breast tumors), and Human epithelial mucin (MUC1) (Breast, ovarian, colon and lung cancers).

Tissue- or lineage-specific cancer antigens also include secreted proteins such as Monoclonal immunoglobulin (Multiple myeloma and plasmacytoma), Immunoglobulin light chains (Multiple Myeloma), □-fetoprotein (Liver carcinoma), Kallikreins 6 and 10 (Ovarian cancer), Gastrin-releasing peptide/bombesin (Lung carcinoma), and Prostate specific antigen (Prostate cancer).

Still other cancer antigens are cancer testis (CT) antigens that are expressed in some normal tissues such as testis and in some cases placenta. Their expression is common in tumors of diverse lineages and as a group the antigens form targets for immunotherapy. Examples of tumor expression of CT antigens include MAGE-A1, -A3, -A6, -A12, BAGE, GAGE, HAGE, LAGE-1, NY-ESO-1, RAGE, SSX-1, -2, -3, -4, -5, -6, -7, -8, -9, HOM-TES-14/SCP-1, HOM-TES-85 and PRAME. Still other examples of CT antigens and the cancers in which they are expressed include SSX-2, and -4 (Neuroblastoma), SSX-2 (HOM-MEL-40), MAGE, GAGE, BAGE and PRAME (Malignant melanoma), HOM-TES-14/SCP-1 (Meningioma), SSX-4 (Oligodendrioglioma), HOM-TES-14/SCP-1, MAGE-3 and SSX-4 (Astrocytoma), SSX member (Head and neck cancer, ovarian cancer, lymphoid tumors, colorectal cancer and breast cancer), RAGE-1, -2, -4, GAGE-I, -2, -3, -4, -5, -6, -7 and -8 (Head and neck squamous cell carcinoma (HNSCC)), HOM-TES14/SCP-1, PRAME, SSX-1 and CT-7 (Non-Hodgkin's lymphoma), and PRAME (Acute lymphoblastic leukemia (ALL), acute myelogenous leukemia (AML) and chronic lymphocytic leukemia (CLL)).

Other cancer antigens are not specific to a particular tissue or cell lineage. These include members of the carcinoembryonic antigen (CEA) family: CD66a, CD66b, CD66c, CD66d and CD66e. These antigens can be expressed in many different malignant tumors and can be targeted by immunotherapy.

Still other cancer antigens are viral proteins and these include Human papilloma virus protein (cervical cancer), and EBV-encoded nuclear antigen (EBNA)-1 (lymphomas of the neck and oral cancer).

Still other cancer antigens are mutated or aberrantly expressed molecules such as but not limited to CDK4 and beta-catenin (melanoma).

Still other cancer antigen may be selected from the group consisting of MART-1/Melan-A, gp100, adenosine deaminase-binding protein (ADAbp), FAP, cyclophilin b, colorectal associated antigen (CRC)——C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The cancer antigen may also be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). In still another embodiment, the cancer antigen is selected from the group consisting of GAGE-1, GAGE-2, GAGE-3, GAGE-4, GAGE-5, GAGE-6, GAGE-7, GAGE-8, GAGE-9. And in yet a further embodiment, the cancer antigen is selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, P-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Subjects

A “subject at risk” of developing an infection as used herein is a subject who has any risk of exposure to an infectious microorganism or pathogen, e.g. someone who is in contact with an infected subject or who is traveling to a place where a particular microorganism is prevalent. For instance, a subject at risk may be a subject who is planning to travel to an area where a particular microorganism is found or it may even be any subject living in an area where a microorganism has been identified. These subjects include those that have a general risk of exposure to a microorganism, e.g., influenza, as well as subjects that are considered to be at specific risk of developing an infectious disease because of medical or environmental factors, that expose them to a particular microorganism.

The invention also contemplates treatment of subjects having or at risk of developing cancer, as such subject may benefit from the induction of an immune response to a cancer antigen. A subject having a cancer is a subject that has detectable cancerous cells. A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

The cancer may be but is not limited to basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; acute myeloid leukemia; acute lymphoid leukemia, chronic myeloid leukemia, chronic lymphoid leukemia, leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

Secondary Agents

The compositions and/or methods of the invention can be used with one or more secondary agents such as but not limited to anti-microbial agents or anti-cancer agents. These agents may be administered substantially simultaneously with the compositions of the invention (i.e., concurrently or within minutes of each other), or they may be spaced apart in time (e.g., hours, days, weeks, etc.). The secondary agents may be administered before, during, or after administration of the compositions of the invention. The conjugates of the invention may be formulated together with one or more secondary agent, in some embodiments.

An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. One type of anti-microbial agent is an antibiotic.

As used herein, antibiotics are compounds that kill or inhibit the growth or function of bacteria. They may or may not be obtained from naturally occurring sources. Antibiotics which are effective for killing or inhibiting a wide range of bacteria are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics.

Antibiotics are sometimes classified based on their primary mode of action. In general, antibiotics are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring which inhibits the last step of peptidoglycan synthesis. β-lactam antibiotics can be synthesized or natural. The natural antibiotics are generally produced by two groups of fungi, penicillium and cephalosporium molds. The β-lactam antibiotics produced by penicillium are the natural penicillins, such as penicillin G or penicillin V. These are produced by fermentation of penicillium chrysogenum. The natural penicillins have a narrow spectrum of activity and are generally effective against streptococcus, gonococcus, and staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulamic acids and sulbactam. The β-lactamase inhibitors do not have anti-microbial action but they function to inhibit penicillinase, thus protecting the semi-synthetic penicillin from degradation.

Another type of β-lactam antibiotic is the cephalolsporins. Cephalolsporins are produced by cephalolsporium molds, and have a similar mode of action to penicillin. They are sensitive to degradation by bacterial β-lactamases, and thus are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis. These antibiotics, produced by bacillus species, prevent cell wall growth by inhibiting the release of muropeptide subunits or peptidoglycan from the molecule that delivers the subunit to the outside of the membrane. Although bacitracin is effective against gram-positive bacteria, its use is limited in general to topical administration because of its high toxicity.

Carbapenems are another broad spectrum β-lactam antibiotic, which is capable of inhibiting cell wall synthesis. Examples of carbapenems include, but are not limited to, imipenems. Monobactems are also broad spectrum β-lactam antibiotics, and include, euztreonam. An antibiotic produced by streptomyces, vancomycin, is also effective against gram-positive bacteria by inhibiting cell membrane synthesis.

Another class of antibiotics are cell membrane inhibitors. These compounds disorganize the structure or inhibit the function of bacterial membranes. Alteration of the cytoplasmic membrane of bacteria results in leakage of cellular materials from the cell. Compounds that inhibit or interfere with the cell membrane cause death of the cell because the integrity of the cytoplasmic and outer membranes is vital to bacteria. An example of a cell membrane inhibitor is Polymyxin, produced by Bacillus polymyxis. Polymyxins interfere with membrane function by binding to membrane phospholipids. Polymyxin is effective mainly against Gram-negative bacteria and is generally used in severe Pseudomonas infections or Pseudomonas infections that are resistant to less toxic antibiotics. It is also used in some limited instances topically.

Other cell membrane inhibitors include Amphotericin B and Nystatin produced by the bacterium Streptomyces which are also anti-fungal agents, used predominantly in the treatment of systemic fungal infections and Candida yeast infections respectively. Imidazoles, produced by the bacterium Streptomyces, are another class of antibiotic that is a cell membrane inhibitor. Imidazoles are used as bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Some antibiotics are protein synthesis inhibitors. These compounds prevent bacteria from synthesizing structural proteins and enzymes and thus cause inhibition of bacterial cell growth or function or cell death. In general these compounds interfere with the processes of transcription or translation. Anti-bacterial agents that block transcription include but are not limited to Rifampins, produced by the bacterium Streptomyces and Ethambutol, a synthetic chemical. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Antibiotics which block translation interfere with bacterial ribosomes to prevent mRNA from being translated into proteins. In general this class of compounds includes but is not limited to tetracyclines, chloramphenicol, the macrolides (e.g. erythromycin) and the aminoglycosides (e.g. streptomycin).

Some of these compounds bind irreversibly to the 30s ribosomal subunit and cause a misreading of the mRNA, e.g., the aminoglycosides. The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant staphylococci.

Another type of translation inhibitor anti-bacterial agent is the tetracyclines. The tetracyclines bind reversibly to the 30s ribosomal subunit and interfere with the binding of charged tRNA to the bacterial ribosome. The tetracyclines are a class of antibiotics, produced by the bacterium Streptomyces, that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease.

Antibiotics such as the macrolides bind reversibly to the 50s ribosomal subunit and inhibit elongation of proteins by peptidyl transferase or prevent the release of uncharged tRNA from the bacterial ribosome or both. The macrolides contain large lactone rings linked through glycoside bonds with amino sugars. These compounds include erythlomycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70S ribosome inhibiting the bacterial enzyme peptidyl transferase thereby preventing the growth of the polypeptide chain during protein synthesis. Chloramphenicol can be prepared from Streptomyces or produced entirely by chemical synthesis.

Some antibiotics disrupt nucleic acid synthesis or function, e.g., bind to DNA or RNA, so that their messages cannot be read. These include but are not limited to quinolones and co-trimoxazole; both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the beta subunit of the polymerase and blocks the addition of the first nucleotide which is necessary to activate the polymerase, thereby blocking mRNA synthesis.

Another class of antibiotics is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g. gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Anti-mycobacterials include Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate and Trecator-SC (Ethionamide).

Antiviral agents are compounds which prevent infection of cells by viruses or replication of the virus within the cell. There are many fewer antiviral drugs than antibiotics because the process of viral replication is so closely related to DNA replication within the host cell that nonspecific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immunoglobulin therapy and hyper-immunoglobulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immuno-compromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Another type of immunoglobulin therapy is active immunization. This involves the administration of antibodies or antibody fragments to viral surface proteins. Two types of vaccines which are available for active immunization of hepatitis B include serum-derived hepatitis B antibodies and recombinant hepatitis B antibodies. Both are prepared from HBsAg. The antibodies are administered in three doses to subjects at high risk of infection with hepatitis B virus, such as health care workers, sexual partners of chronic carriers, and infants.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Some forms of aspergillus can be treated with prednisone, disodium chromoglycat, nystatin, amphotericin B, itraconazole, or voriconazole. Blastomycosis is treated by amphotericin B, hydroxystilbamidine, itraconazole and/or voriconazole. Candidiasis is treated with nystatin, amphoterizin B, 5-fluorocytosine, fluconazole, itraconazole and/or voriconazole. Chromoblastomycosis is treated with itraconazole and/or terbinafine. Coccidioidomycosis is treated with amphoterizin B, itraconazole, fluconazole, ketaconazole, and/or voriconazole. Cryptococcosis is treated with amphoterizin B and/or 5-fluorocytosine and in some cases fluconazole. Drugs useful for treating mycotit keratitis include pimaricin and fluconazole. Occulomycosis is treated with amphoterizin B. Fungal infections of the hair, nail, and skin can be treated with itraconazole, turbinifine, amphoterizin B, gentian violet, resorcin, iodine, nystatin, thiabendazole, and glutarardehyde. Piedra can be treated with keratolytic agents, mild fungicides, fluconazole, and itraconazole. The tineas can be treated with keratolytic agents, intraconazole, turbinifine, tolnaftate, chlotrimazole, miconazole, econazole, and ketaconzole. Histoplasmosis (capsulati and duboisii) can be treated with amphoterizin B, itraconazole or voriconazole. If the subject being treated has AIDS, fluconazole is usually used. Histoplasmosis duboisii once it becomes disseminated can be treated with amphoterizin B, itraconazole, fluconazole, and voriconazole. The mycetomas can be treated with ketoconazole. Paracoccidioidomycosis can be treated with amphoterizin B and sulfonamides are generally used to treat the disease. Phaeohyphomycosis is treated with amphoterizin B and phyfluorocytozine or intaconazole. Rhinosporidiosis is treated with amphoterizin B. Sporotrichosis is treated with oral potassium iodide, amphoterizin B, or 5-fluorocytozine. Zygomycosis is treated with potassium iodide and/or amphoterizin B.

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

When used in subjects having or at risk of developing cancer, the compositions of the invention may be used together with anti-cancer agents. Anti-cancer agents are agents that slow or inhibit the growth of cancer cells, and/or alleviate partially or completely symptoms related to the cancer.

The anti-cancer agents may be enzyme inhibitors selected from the group consisting of a tyrosine kinase inhibitor, a CDK inhibitor, a MAP kinase inhibitor, and an EGFR inhibitor. The tyrosine kinase inhibitor includes but is not limited to Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, and HDBA (2-Hydroxy5-(2,5-Dihydroxybenzylamino)-2-hydroxybenzoic acid. The CDK inhibitor includes but is not limited to p21, p27, p57, p15, p16, p18, and p19. The MAP kinase inhibitor includes but is not limited to KY12420 (C₂₃H₂₄O₈), CNI-1493, PD98059, 4-(4-Fluorophenyl)-2-(4-methylsulfinyl phenyl)-5-(4-pyridyl)1H-imidazole. The EGFR inhibitor includes but is not limited to Tarceva™(OSI-774), Iressa (ZD1839), WHI-P97 (quinazoline derivative), LFM-A12 (leflunomide metabolite analog), AG1458.

Other anti-cancer agents can be categorized as DNA damaging agents and these include topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy.

Important anticancer agents include but are not limited to Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Gemcitabine Hydrochloride; Gemtuzumab Ozogamicin; Gold Au 198; Goserelin Acetate; Guanacone; Hydroxyurea; Idaribicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride: Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751: linomide; sulfur mustard; nitrogen mustard (mechlor ethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis(platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl)retinamide; 13-cis retinoic acid; 3-Methyl TTNEB; 9-cis retinoic acid; fludarabine(2-F-ara-AMP); 2-chlorodeoxyadenosine (2-Cda), 20-epi-1,25dihydroxyvitamin D3; 5-ethynyluracil; abiraterone; aclarubicin; acylfulvene; adecypenol; adozelesin; aldesleukin; ALL-TK antagonists; altretamine: ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A₂; bleomycin B₂; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin: crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R═H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonaclotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhihitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Other anti-cancer agents include Antiproliferative agents (e.g., Piritrexim Isothionate), Antiprostatic hypertrophy agent (e.g., Sitogluside), Benign prostatic hyperplasia therapy agents (e.g., Tamsulosin Flydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine 1 125; Thyroxine 1 131; Tolpovidone 1 131; Triolein 1 125; Triolein 1 131.

Another category of anti-cancer agents is anti-cancer Supplementary Potentiating Agents, including Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Particularly important anticancer agents are those selected from the group consisting of annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-Cl-2′deoxyadenosine; Fludarabine-PO₄; mitoxantrone; mitozolomide; Pentostatin; Tomudex, taxanes (e.g., paclitaxel and docetaxel), and annonaceous acetogenin.

Therapeutic Packages

The invention also includes therapeutic packages for use in vaccination or in the treatment of subjects. Such packages will comprise at least one container containing the conjugates described above along with instructions for administering the compositions to a subject. The conjugates, along with other vaccine components may either be ready for immediate administration or they may be in a lyophilized or concentrated state, requiring reconstitution or dilution before use. In a preferred embodiment, the conjugates will be used for injection and will be in a finished pharmaceutical container such as an injection ampoule or vial. Alternatively, vaccine compositions may be supplied in prefilled disposable syringes, each containing an amount of vaccine suitable for administration to a single subject.

Advantages

The vaccine compositions described herein may be used to generate a strong immune response using antigens that are poorly immunogenic when administered in an unconjugated form. Antigenic oligosaccharides, lipids, and nucleic acids such as those present in bacteria tend to be less variable than their protein counterparts and may even be common between species. Thus, one vaccine may be able to target more than one pathogen. Another advantage is that the immune response obtained using the conjugates is rapid (e.g., detected within six days) and, as a result, these conjugates may be effective for subjects already infected with a pathogen or already having a disease.

### EXAMPLES

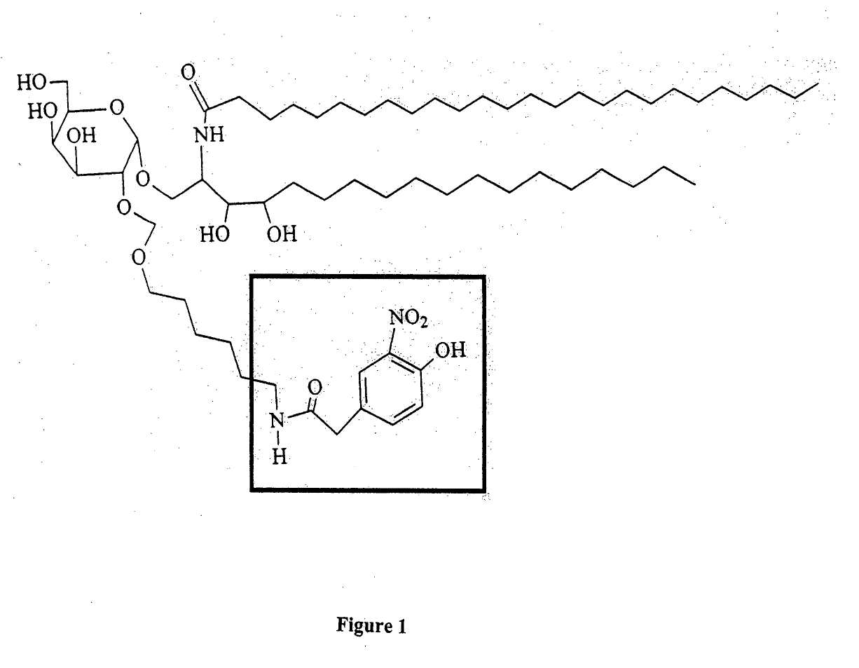

There are a growing number of recently described lipid antigens presented by the MHC I-like CD1d antigen presenting molecule that are recognized by a specialized subset of rapid-responding T cells, the NK T cells. Isotype-switched antibodies to lipid antigens have been isolated following infection or during autoimmune disease. This suggests that NK T cells may be helping B cells improve their response to antigens presented by CD1d. To investigate this possibility, a haptenated lipid antigen (NP-α-GalCer, see FIG. 1) which will be recognized by NP-specific B cells as well as α-GalCer-specific NK T cells we synthesized.

In vivo immunization of mice with NP-α-GalCer stimulated a strong IgG antibody response specific for NP. This antibody production was CD1d and Jα281 NK T cell dependent. Specific antibody was also produced in response to NP-α-GalCer more rapidly and to a higher titer than antibody was produced in response to challenge with a haptenated protein antigen, NP-KLH, even when the protein was administered in combination with a general immune-stimulating adjuvant. NP-α-GalCer also induced a higher titer and faster response than α-GalCer mixed with, but not linked to, NP-KLH. The NK T cell-dependent nature of this response provided evidence of NK T cell help for a B cell antibody response. The mixing versus linked experiments suggested the response may involve cognate presentation of the antigen by CD1d on the NP-specific B cell.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A pharmaceutical composition comprising:
a) a lipid, glycan, nucleic acid, or peptide antigen conjugated to a compound of Formula I: 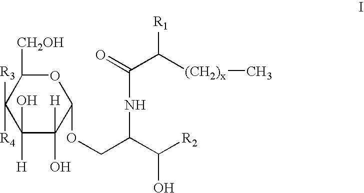
wherein:
R₁ is: H or OH;
R₂ is: ——CH₂(CH₂)_{Y}CH₃; ——CH(OH)(CH₂)_{Y}CH₃; ——CH═CH(CH₂)_{Y}CH₃;
or ——CH(OH)(CH₂)_{Y}CH(CH₃)CH₂CH₃, where Y=an integer from 5-17;
R₃ and R₄ are: H or OH such that when R₃ is H, R₄ is OH and when R₃ is OH, R₄ is H; and
X is an integer from 7-25; and
b) a pharmaceutically acceptable carrier.

2. The pharmaceutical composition of claim 1, wherein R₃ is OH.

3. The pharmaceutical composition of claim 1, wherein said antigen is derived from a pathogenic microorganism selected from the group consisting of Hemophilus influenza type B, Neisseria meningitides, Salmonella typhi, and Streptococcus pneumoniae.

4. The pharmaceutical composition of claim 1, wherein said antigen is a lipid.

5. The pharmaceutical composition of claim 4, wherein R₃ is OH and said lipid is conjugated to the sugar portion of the compound of Formula I.

6. The pharmaceutical composition of claim 5, wherein R₂ is either: ——CH(OH)(CH₂)_{Y}CH₃; or —CH═CH(CH₂)_{Y}CH₃; Y=10-17 and X=15-25.

7. The pharmaceutical composition of claim 6, wherein R₁═H; R₂ is: ——CH(OH)(CH₂)_{Y}CH₃; Y=13 and X=23.

8. A therapeutic package comprising the pharmaceutical composition of claim 1 in a finished injection ampoule, vial or syringe together with instructions for the administration of said pharmaceutical composition to a subject to induce an immune response.

9. The therapeutic package of claim 8, wherein R₃ in said pharmaceutical composition is OH.

10. The therapeutic package of claim 8, wherein said antigen is derived from a pathogenic microorganism selected from the group consisting of Hemophilus influenza type B, Neisseria meningitides, Salmonella typhi, and Streptococcus pneumoniae.

11. The therapeutic package of claim 8, wherein said antigen in said pharmaceutical composition is a lipid.

12. The therapeutic package of claim 11, wherein R₃ in said pharmaceutical composition is OH and said lipid is conjugated to the sugar portion of the compound of Formula I.

13. The therapeutic package of claim 12, wherein R₂ in said pharmaceutical composition is either ——CH(OH)(CH₂)_{Y}CH₃ or —CH═CH(CH₂)_{Y}CH₃, and wherein Y=10-17 and X=15-25.

14. The therapeutic package of claim 13, wherein, in said pharmaceutical composition R₁═H, R₂ is —CH(OH)(CH₂)_{Y}CH₃, Y=13, and X=23.

15. A method of inducing the production of antibodies against an antigen comprising administering to a subject capable of antibody production an effective amount of the pharmaceutical composition of claim 1.

16. The method of claim 15, wherein R₃ in said pharmaceutical composition is OH.

17. The method of claim 15, wherein said antigen in said pharmaceutical composition is derived from a pathogenic microorganism selected from the group consisting of Hemophilus influenza type B, Neisseria meningitides, salmonella typhi, and Streptococcus pneumoniae.

18. The method of claim 15, wherein said antigen in said pharmaceutical composition is a lipid.

19. The method of claim 18, wherein R₃ in said pharmaceutical composition is OH and said lipid is conjugated to the sugar portion of the compound of Formula I.

20. The method of claim 19, wherein R₂ in said pharmaceutical composition is either ——CH(OH)(CH₂)_{Y}CH₃ or —CH═CH(CH₂)_{Y}CH₃, and wherein Y=10-17 and X=15-25.

21. The method of claim 20, wherein, in said pharmaceutical composition R₁═H, R₂ is —CH(OH)(CH₂)_{Y}CH₃, Y=13, and X=23.

22. A method of treating a subject that has been infected with a pathogen comprising administering to said subject an effective amount of the pharmaceutical composition of claim 1, wherein said antigen induces an immune response against said pathogen.

23. The method of claim 22, wherein R₃ in said pharmaceutical composition is OH.

24. The method of claim 22, wherein said antigen is derived from a pathogenic microorganism selected from the group consisting of Hemophilus influenza type B, Neissenia meningitides, Salmonella typhi, and Streptococcus pneumoniae.

25. The method of claim 22, wherein said antigen in said pharmaceutical composition is a lipid.

26. The method claim of 25, wherein R₃ in said pharmaceutical composition is OH and said lipid is conjugated to the sugar portion of the compound of Formula I.

27. The method of claim 26, wherein R₂ in said pharmaceutical composition is either ——CH(OH)(CH₂)_{Y}CH₃ or —CH═CH(CH₂)_{Y}CH₃, and wherein Y=10-17 and X=15-25.

28. The method of claim 27, wherein, in said pharmaceutical composition R₁═H, R₂ is ——CH(OH)(CH₂)_{Y}CH₃, Y=13, and X=23.
